(19) United States
(12) Patent Application Publication
(10) Pub. No.: US 2006/0009770 A1
(43) Pub. Date: Jan. 12, 2006

(54) Bone plate and bone screw system

(76) Inventors: Andrew Speirs, Arnprior (CA); Raymond Desjardins, Arnprior (CA)

Correspondence Address: SHUGHART THOMSON & KILROY, PC, 120 WEST 12TH STREET, KANSAS CITY, MO, 64105, US

(21) Appl. No.: 10/992,020

(22) Filed: Nov. 18, 2004

(30) Foreign Application Priority Data
Nov. 18, 2003 (CA) 2.449,883

(51) Int. Cl. A61B 17/56

(52) U.S. Cl. 606/69

(57) ABSTRACT

A bone screw having a head portion and a threaded shaft extending therefrom is described. The head portion has a top surface with a drive means formed therein coaxial with the threaded shaft and a profiled portion between the top surface and the threaded shaft. The profiled portion of the body is provided with a circumferential groove on its surface, substantially coaxial with the threaded shaft.

### FIELD OF THE INVENTION

The present invention relates to the field of orthopedic implants, and in particular, provides a bone plate and bone screw system in which the bone screws, when fully screwed into place, become locked in position relative to the bone plate.

### BACKGROUND OF THE INVENTION

In traditional bone plate/bone screw systems, the plate is shaped to fit a particular bone or type of bone (e.g., a long bone) and a series of holes are pre-formed through the plate, so that the plate may be fastened to a bone that requires repair by two or more bone screws. It is typical that the plates are partially spherically counterbored around the screw holes, and the bore screws will have heads with substantially matching profiles, so that the screw heads will, when driven home, be countersunk into the surface of the bone plate and may be angled somewhat relative to the bone plate by virtue of the partially spherically counterbore and complementary screw head.

In order to effectively prevent bone plates from shifting under stress after implantation on a bone, it has been proposed to provide a bone plate/bone screw system that fixes the angle of a bone screw relative to the bone plate upon implantation, and prevents the screw from moving or “toggling” in the bone.

There have been several systems proposed to accomplish the goal of angular fixation. To begin with, it will be understood that the screw head of the bone screw should be countersunk into the bone plate in order to lessen irritation to the surrounding tissue. The screw head, to be countersunk, must be round, and match the profile of the counterbore. If the screw head is round and partially spherical in profile, it will have a natural tendency to toggle, or shift angularly, in the screw hole. A beveled screw head can also toggle somewhat, because generally speaking, the taper must be at a fairly high angle, relative to the longitudinal axis of the screw, in order that the head not be too deep, which would require an overly thick bone plate for countersinking purposes. it is, moreover, extremely important that a bone screw, once tightened, not become loose. A loose bone plate can prevent proper healing of a bone, and may require surgery for removal.

One system that has been proposed to fix the angle of a bone screw and lock it in place is shown in U.S. Pat. No. 6,383,186. In that patent, a system is described in which a locking mechanism is built into the bone plate. The locking mechanism, which functions to keep the bone screw at a set angle and prevent it from loosening the locking mechanism, is a rotatable disc-like element that rotates within or screws into a fitting on the bone plate, to interlock with, or cover, the head of a screw that has been driven in. This system is, then, fairly complex and relies on locking elements that are separate and distinct from the bone screws. The locking elements must either be built into the plate, or must screw onto the plate.

Another way to fix the angle of a bone screw relative to a plate is shown in U.S. Pat. No. 5,607,428, which shows a threaded fixation device for insertion into a hole on a bone plate, to accept a bone screw therethrough. The fixation device has a threaded bore, which may be at an angle to the bone plate, into which the bone screw is screwed. The screw, then, must have fairly coarse threads along most of its length to engage bone, but at its upper extremity, is provided with finer threads to screw into the fixation device. This is not practical, because to engage the fine screw threads which are set close together, the screw must be turned a number of times without substantially advancing in the bone. This may damage the bone.

The present invention, on the other hand, provides a bone plate/screw system in which predetermined screw angles are bored into a bone plate, and locking means on the screw are provided, to interfit with grooves formed in the profiled counterbore on the plate. The use of a locking ring, also called a snap-ring, in an orthopedic plate assembly is shown in U.S. Pat. No. 5,876,402, but in that patent, the ring acts more as a cap than a lock, because it is not captive on the screw. This is because the screw shown in U.S. Pat. No. 5,876,402 is a swiveling screw with a ball-joint head.

In a broad aspect, then, the present invention relates to a bone screw having a head portion and a threaded shaft extending therefrom, said head portion having a top surface with a drive means formed therein coaxial with said threaded shaft, and a profiled portion between said top surface and said threaded shaft wherein said profiled portion of said body is provided with a circumferential groove on its surface, substantially coaxial with said threaded shaft.

In another broad aspect, then, the present invention relates to a bone screw having a head portion and a threaded shaft extending therefrom, said head portion having a top surface with drive means formed therein, coaxial with said threaded shaft, and a profiled portion between said top surface and said threaded shaft, said profiled portion being dimensioned to fit snugly and substantially unmovably in a complementary screw head accommodating depression in a bone plate.

In a further broad aspect, then, the present invention relates to a bone plate far use with a bone screw as described above, including at least one screw hole having a cross-section shaped to accommodate the profiled head of a said screw, and a circumferential groove formed in said countersink, alignable with the circumferential groove on said head.

### DESCRIPTION OF THE DRAWINGS

FIG. 1 is an oblique cross-section of a portion of a bone plate/bone screw system of the present invention;

FIG. 2 is a cross-section of a portion of a bone plate/bone screw system of the present invention;

FIG. 3 is a detail view of a bone screw head of the present invention;

FIG. 4 is a cross-sectional view of an alternative screw head shape;

FIG. 5 is a cross-sectional view of another alternative screw head shape;

FIG. 6 is a cross-sectional view, similar to the view of FIG. 2, showing the bone screw set at an angle to the bone plate;

FIG. 7 is a perspective view of a bone plate/bone screw combination of the present invention, in further combination with a prior art slot and countersink screw hole.

### DESCRIPTION OF THE PREFERRED EMBODIMENT

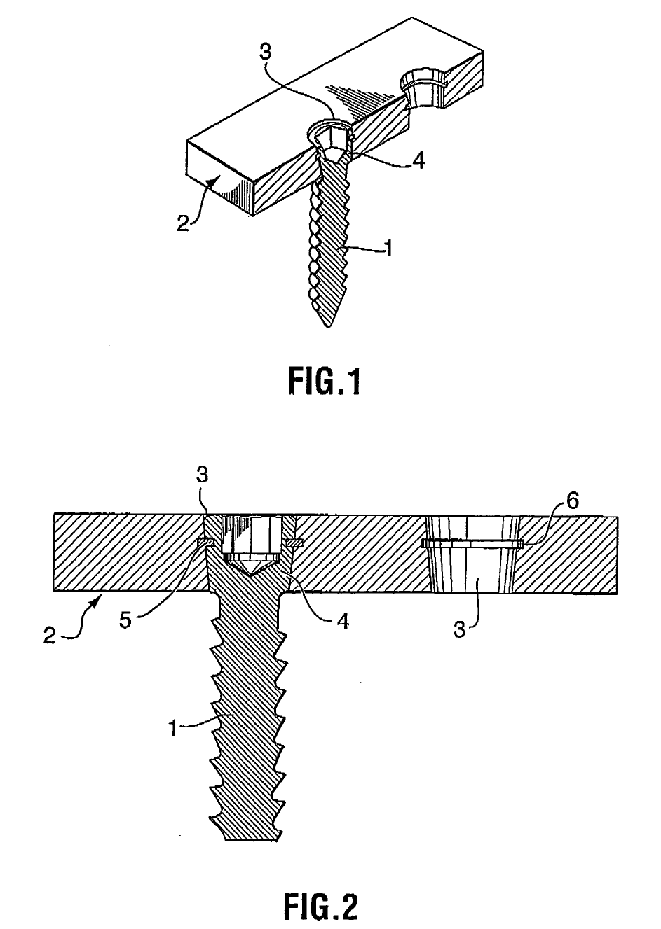

As can be seen in FIGS. 1 and 2, the present invention provides a bone screw 1 in combination with a bone plate 2. The bone plate 2 shown in FIG. 1 is a simple rectangle for illustrating purposes only. In fact, bone plates are typically produced in a variety of shapes for a variety of applications. The bone plate essentially is a fairly low profile metallic piece, with two or more screw holes formed in it. It may be fairly long, for use in repairing long bones such as the tibia, or it may be short. The plate may be narrow, i.e., one screw hole wide, or it may span several screw holes if designed for repair of wide flat bones such as the pelvis or scapula. It may be T-shaped, L-shaped, spoon shaped, or be pre-contoured or bent into a specific shape by a surgeon. It will be appreciated, then, that the small rectangular representation of a bone plate shown in this application is by no means meant to be exhaustive. Moreover, it will be understood that it is not necessary, in order to gain most of the benefits of a preferred embodiment of the present invention, to adapt all screw holes in a bone plate according to the present invention to accept the bone screws of the present invention. Many benefits can be obtained by using even only one screw of the present invention on a bone plate. For instance, one of the benefits of the screws of a preferred embodiment of the present invention is that they will not loosen, and partially back up because they are locked in a driven-in position. This will prevent a bone plate from coming loose on a bone being repaired if even one screw according to the present invention is used. Furthermore, the positioning of screw holes on a plate may be co-linear, or may be dictated by anatomical considerations.

Referring, then, to FIG. 1, the two principle features of the present invention are illustrated. In the preferred embodiment of the present invention, the screw head 4 is a highangle, self locking design such as the Morse taper illustrated which, when driven into a complementary shaped screw hole 3, will lock itself in position, at a fixed angle relative to the bone plate, by friction.

In order to ensure that the bone screw 1 remains in the hole 3 and does not back out or loosen substantially, the present invention employs a snap-ring 5 fitted around a groove 8 formed in the head 4 of the screw 1, which is compressible when the screw is being driven into place, and then will snap into a groove 6 formed in the screw hole. The snap-ring is preferably formed from a high tensile metallic material such as stainless steel, and is fabricated as a washer with a gap cut in it. It is expanded outwardly to be placed in position on a screw, and then squeezed together to be fit inside a screw hole on the head of a screw. When the snap-ring is aligned with the groove 6 formed in the screw hole, it will snap into the groove and lock the screw into position relative to the screw hole.

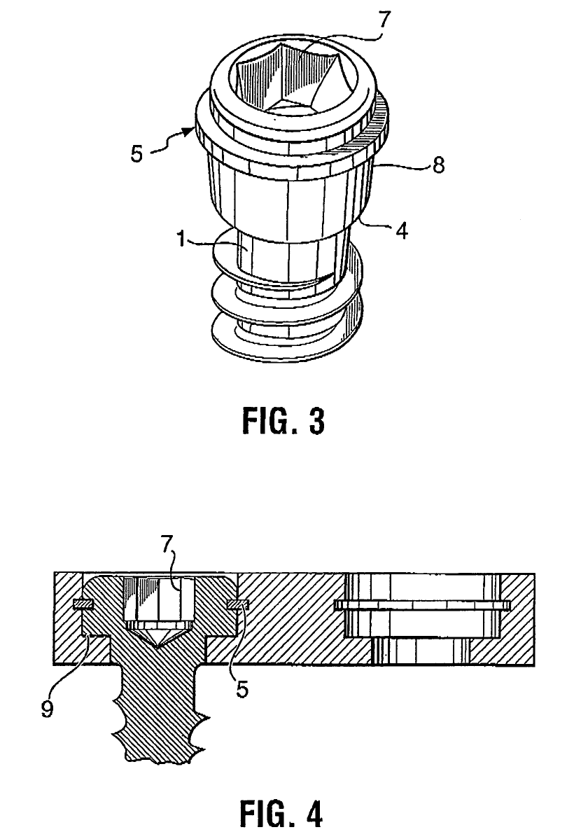

Referring to FIG. 3, which shows a screw head with a snap-ring in place thereon, it will be observed that a drive socket 7, such as the hexagonal drive socket 7 shown, is formed in the top surface of the screw head. Any desired shape of drive socket may be used, of course, hexagonal being preferred because of reliability and prevalence of hexagonal shaped drive tools currently in use.

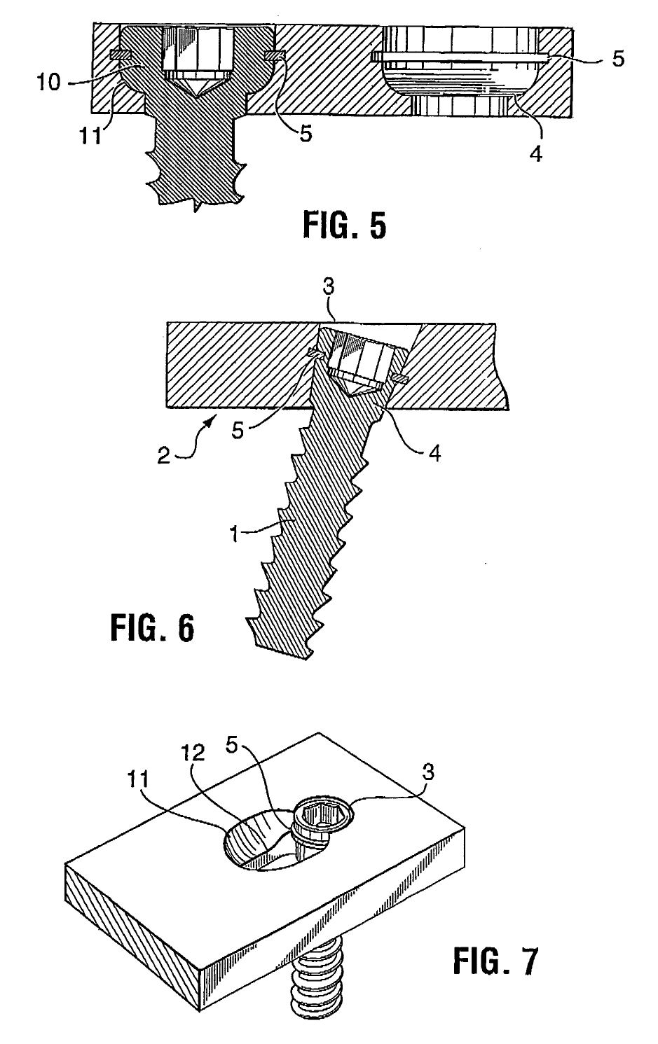

In FIGS. 4 and 5, alternative shapes of screw holes are illustrated. In FIG. 4, a squared-off counterbore is shown, which would ordinarily not provide any locking of the screw head in the screw hole. That is, the screw head 3 is a fairly snug fit in the counterbore, but the counterbore 9 is not ordinarily designed to hold the screw head against loosening or backing out, nor is it well adapted to maintain a fixed angle between the screw and the bone plate. With the addition, however, of the snap-ring 5 of the present invention, located in the annular groove formed on the screw head, the screw is locked in position in the counterbore when the snap-ring engages the groove formed in the counterbore. This will lock the bone screw angularly and axially relative to the bone plate. A similar arrangement is shown in FIG. 5, which shows a bone screw with a generally spherically shaped head 10. Such heads are desirable because they seat well against the bottom of the countersink 11 which will also be spherical in shape. It will be appreciated, though, that a screw head with a spherical shape will normally have a tendency to toggle angularly relative to the bone plate. Using the snap-ring of the present invention with such a spherically shaped screw head will provide the benefits associated with a spherical screw head, but will permit the screw head to be angularly locked relative to the bone plate.

In FIG. 7, an alternative embodiment of a bone plate for use with the bone screw of the present invention is illustrated. The screw hole in such a bone plate may be formed in two distinct, but joined portions. A first, conventional portion 12 is provided, with an elongated screw hole and a generally spherical countersink 11. This portion of the hole will permit the use of a conventional bone screw with a generally spherical head, to be used, with the bone screw driven in at any required angle. At one end of the elongated hole, though, a screw hole according to the present invention, preferably with a snap-ring groove formed on the countersink, is provided. Therefore, if surgically appropriate, a screw can be driven through that part of the hole and locked in place angularly and axially to help to keep the bone plate in place with a minimum number of screws. In this regard, it must be borne in mind that it is always desired to use a minimum number of bone screws, to prevent removal of bone, prevent possible damage to bone, promote healing and minimize the number of possible infection sites.

Therefore, it will be understood that using one screw according to the present invention in a screw hole according to the present invention will give the benefit of having the bone plate remain firmly in place. Using a pair of bone screws according to the present invention, in screws holes according to the present invention, will provide even greater benefits since it will prevent angular displacement of the bone plate relative to the bone at each end of a bone plate to stabilize a fracture site.

FIG. 6 illustrates an alternative embodiment of a screw hole according to the present invention. That is, the screw hole may be formed at an angle relative to the bone plate. This is advantageous for certain bone plates having predetermined uses, such as those designed to stabilize fractures near the head of a femur, where it is known that one or more screws will be driven in at an angle to the bone plate.

1. A bone screw having a head portion and a threaded shaft extending therefrom, said head portion having a top surface with a drive means formed therein coaxial with said threaded shaft, and a profiled portion between said top surface and said threaded shaft wherein said profiled portion of said body is provided with a circumferential groove on its surface, substantially coaxial with said threaded shaft.

2. A bone screw as claimed in claim 1, wherein said groove has a resilient, circumferentially compressible ring captive therein.

3. A bone screw as claimed in claim 2, wherein said resiliently compressible ring is a metallic snap-ring.

4. A bone screw having a head portion and a threaded shaft extending therefrom, said head portion having a top surface with drive means formed therein, coaxial with said threaded shaft, and a profiled portion between said top surface and said threaded shaft, said profiled portion being dimensioned to fit snugly and substantially unmovably in a complementary screw head accommodating depression in a bone plate.

5. A bone screw as claimed in claim 2, wherein said resiliently compressible ring is made from a polymeric material.

6. A bone screw as claimed in claim 1, wherein the head of said screw is profiled in a continuous taper.

7. A bone screw as claimed in claim 4, wherein the head of said screw is profiled in a continuous taper.

8. A bone screw as claimed in claim 1, wherein the head of said screw is profiled in a beveled taper.

9. A bone screw as claimed in claim 4, wherein the head of said screw is profiled in a beveled taper.

10. A bone screw as claimed in claim 1, wherein the head of said screw is partially spherically profiled.

11. A bone screw as claimed in claim 4, wherein the head of said screw is partially spherically profiled.

12. A bone plate for use with a bone screw as claimed in claim 1, including at least one screw hole having a cross-section shaped to accommodate the profiled head of a said screw, and a circumferential groove formed in said countersink, allignable with the circumferential groove on said head.

13. A bone plate for use with a bone screw as claimed in claim 4, including at least one screw hole having a cross-section shaped to accommodate the profiled head of a said screw, and a circumferential groove formed in said countersink, allignable with the circumferential groove on said head.
